SEQUENCE LISTING (10 sequences)

SEQ ID NO: 1 - DNA, 2671 bp, homo sapiens
aagatgggtctctaccgcatccgcgtgtccactggggcctcgctctatgccggttccaacaaccaggtgcagctgtggctggtcggccagcacggggaggcggcgctcgggaagcgactgtggcccgcacggggcaaggagacagaactcaaggtggaagtaccggagtatctggggccgctgctgtttgtgaaactgcgcaaacggcacctccttaaggacgacgcctggttctgcaactggatctctgtgcagggccccggagccggggacgaggtcaggttcccttgttaccgctgggtggagggcaacggcgtcctgagcctgcctgaaggcaccggccgcactgtgggcgaggaccctcagggcctgttccagaaacaccgggaagaagagctggaagagagaaggaagttgtaccggtggggaaactggaaggacgggttaattctgaatatggctggggccaaactatatgacctccctgtggatgagcgatttctggaagacaagagagttgactttgaggtttcgctggccaaggggctggccgacctcgctatcaaagactctctaaatgttctgacttgctggaaggatctagatgacttcaaccggattttctggtgtggtcagagcaagctggctgagcgcgtgcgggactcctggaaggaagatgccttatttgggtaccagtttcttaatggcgccaaccccgtggtgctgaggcgctctgctcaccttcctgctcgcctagtgttccctccaggcatggaggaactgcaggcccagctggagaaggagctggagggaggcacactgttcgaagctgacttctccctgctggatgggatcaaggccaacgtcattctctgtagccagcagcacctggctgcccctctagtcatgctgaaattgcagcctgatgggaaactcttgcccatggtcatccagctccagctgccccgcacaggatccccaccacctccccttttcttgcctacggatcccccaatggcctggcttctggccaaatgctgggtgcgcagctctgacttccagctccatgagctgcagtctcatcttctgaggggacacttgatggctgaggtcattgttgtggccaccatgaggtgcctgccgtcgatacatcctatcttcaagcttataattccccacctgcgatacaccctggaaattaacgtccgggccaggactgggctggtctctgacatgggaattttcgaccagataatgagcactggtgggggaggccacgtgcagctgctcaagcaagctggagccttcctaacctacagctccttctgtccccctgatgacttggccgaccgggggctcctgggagtgaagtcttccttctatgcccaagatgcgctgcggctctgggaaatcatctatcggtatgtggaaggaatcgtgagtctccactataagacagacgtggctgtgaaagacgacccagagctgcagacctggtgtcgagagatcactgaaatcgggctgcaaggggcccaggaccgagggtttcctgtctctttacaggctcgggaccaggtttgccactttgtcaccatgtgtatcttcacctgcaccggccaacacgcctctgtgcacctgggccagctggactggtactcttgggtgcctaatgcaccctgcacgatgcggctgcccccgccaaccaccaaggatgcaacgctggagacagtgatggcgacactgcccaacttccaccaggcttctctccagatgtccatcacttggcagctgggcagacgccagcccgttatggtggctgtgggccagcatgaggaggagtatttttcgggccctgagcctaaggctgtgctgaagaagttcagggaggagctggctgccctggataaggaaattgagatccggaatgcaaagctggacatgccctacgagtacctgcggcccagcgtggtggaaaacagtgtggccatctaagcgtcgccaccctttggttatttcagcccccatcacccaagccacaagctgaccccttcgtggttatagccctgccctcccaagtcccaccctcttcccatgtcccaccctccctagaggggcaccttttcatggtctctgcacccagtgaacacattttactctagaggcatcacctgggaccttactcctctttccttccttcctcctttcctatcttccttcctctctctcttcctctttcttcattcagatctatatggcaaatagccacaattatataaatcatttcaagactagaatagggggatataatacatattactccacaccttttatgaatcaaatatgatttttttgttgttgttaagacagagtctcactttgacacccaggctggagtgcagtggtgccatcaccacggctcactgcagcctcagcgtcctgggctcaaatgatcctcccacctcagcctcctgagtagctgggactacaggctcatgccatcatgcccagctaatatttttttattttcgtggagacggggcctcactatgttgcctaggctggaaataggattttgaacccaaattgagtttaacaataataaaaagttgttttacgctaaagatggaaaagaactaggactgaactattttaaataaaatattggcaaaag

SEQ ID NO: 2 - protein, 661 aa, homo sapiens
MGLYRIRVSTGASLYAGSNNQVQLWLVGQHGEAALGKRLWPARGETELKVEVPEYLGPLLFVKLRKRHLLKDDAWFCNWISVQGPGAGDEVRFPCYRWVEGNGVLSLPEGTGRTVGEDPQGLFQKHREEELEERRKLYRWGNWKDGLILNMAGAKLYDLPVDERFLEDKRVDFEVSLAKGLADLAIKDSLNVLTCWKDLDDFNRIFWCGQSKLAERVRDSWKEDALFGYQFLNGANPVVLRRSAHLPARLVFPPGMEELQAQLEKELEGGTLFEADFSLLDGIKANVILCSQQHLAAPLVMLKLQPDGKLLPMVIQLQLPRTGSPPPPLFLPTDPPMAWLLAKCWVRSSDFQLHELQSHLLRGHLMAEVIVVATMRCLPSIHPIFKLIIPHLRYTLEINVRARTGLVSDMGIFDQIMSTGGGGHVQLLKQAGAFLTYSSFCPPDDLADRGLLGVKSSFYAQDALRLWEIIYRYVEGIVSLHYKTDVAVKDDPELQTWCREITEIGLQGAQDRGFPVSLQARDQVCHFVTMCIFTCTGQHASVHLGQLDWYSWVPNAPCTMRLPPPTTKDATLETVMATLPNFHQASLQMSITWQLGRRQPVMVAVGQHEEEYFSGPEPKAVLKKFREELAALDKEIEIRNAKLDMPYEYLRPSVVENSVAI

SEQ ID NO: 3 - DNA, 3224 bp, homo sapiens
cagcttgcagtagagagctaaactggtcaggaggatggcgaaatgcagggtgagagtatccacgggggaagcctgtggggctggcacatgggacaaagtgtctgtcagcatcgtgggaacccacggagagagccccttagtacctctggaccatctgggcaaggagttcagcgccggtgctgaagaagacttcgaggtgacgcttccccaggacgtaggcactgtgctgatgctgcgagtccacaaagcacccccggaagtgtccctcccgcttatgtctttccgttctgatgcctggttctgccgctggttcgagctggagtggctacctggggctgcactccacttcccctgttatcagtggctggaaggggcgggggagctggtgctgagagagggagcagcaaaggtgtcctggcaagaccatcaccctacactgcaggatcagcgccagaaggagcttgagtccaggcagaagatgtacagctggaagacttacattgaaggttggcctcgctgccttgaccacgagactgtgaaagacttggacctcaacatcaagtactctgcgatgaagaatgccaaactcttctttaaagcccactccgcgtatacggagctgaaagtcaaagggctcctggaccgcacaggactctggaggagtctgagggagatgagaaggctgtttaacttccgcaagactccagcagcagagtatgtgtttgcacactggcaggaagatgccttcttcgcctcccagttcctaaatggcatcaacccggtcctgattcgccgctgtcacagtctcccaaacaacttcccggtcactgatgaaatggtggccccagtgctgggccctggaaccagtctgcaggctgagttggagaagggctccctgttcttggtggatcatggcattctttctggagtccacaccaacatcctcaatggaaagcctcagttctctgcagccccgatgaccctgttacaccagagctcagggtccggacccctgcttcccattgccatccagctcaaacagactcccgggccagacaaccccatcttcctgcccagcgatgacacgtgggactggttgctggccaagacctgggttcgcaattctgagttttacatccatgaggctgtcacacatctgctgcatgcccatctgattccagaagtctttgccttggccacattacgtcagctgcctaggtgtcaccctctcttcaagctattgattcctcacattcggtacacactgcacatcaacacgcttgcccgggagctgctcgttgcccctgggaagttgatagacaagtccacaggccttggcactgggggattctctgacctgataaagagaaacatggagcagctgaactactctgtcctgtgtctccctgaagatatccgagcccgaggtgtggaagacatcccaggctactattaccgagatgatgggatgcagatctggggggcaataaagagctttgtctctgaaatagtcagcatctactatccaagtgacacatccgtccaagatgaccaagagctccaggcctgggtgagggagatcttctctgagggcttcctcggccgagaaagctcaggtatgccctccttgttggatacccgggaagccctggtccagtatatcaccatggtgatattcacctgctcagccaagcatgcagctgtcagttcaggccagttcgactcttgtgtttggatgcccaatctgccacctaccatgcagctaccaccacctacttccaaaggccaggcccggcctgagagtttcatagccacgctcccagcagttaattcgtcaagttatcacatcattgctctctggctgctaagcgcagaacctggggaccaaaggcccctgggccactatccagatgaacacttcacagaggatgccccccggcgaagcgtggctgccttccagagaaagctgatccagatctccaagggcatcagggagaggaaccgaggcctggcactgccctacacctacctggatcctcccctcattgagaacagtgtctccatctaacatcttggagaagacagtcctgtgtgacatatagaactcttgaccatgcctctccaggctaagtccccgtatgcttctcctggacaaccaagccccatcttacacacacacacacacacacacacctaataaaatcgaaacagaaaaacctaaactcccacagaaggcaagatctcacacagcagagagccatccaaatgtttggagaccctgagcttcagctctgattaacggctttgctggtttgctttgctttctattccattaaccatggacggtaacagaaagcacagaaccctggttcactgcacaaagccactgagatctcaccctcacctgacacaaaggcagctatcatacaggcttatcaggaacacaggaatttgtccaatcaaagcctacccactaggtccatcgtgacctacgacctcacactggcatgctttagctttgagaagggattactggagtcaggtacgaagagaaggacaggacgaaggcatggctccatgtggaagaacatatctgctcttccagatgaccagggtagctcacagccatgtgtcattctaactccagaggtctctagtggccatgaagactccaggcattcaggggatataccagtagacaccaaaattatactttttaagagagaggaatgggctggagagatggctcagcggttaagagcactgactgctcttccagagatcctgagttcaattcccagcaaccacatggtggctcacaaccatctgtaatgggattcgatgccctcttctggcgtgtctgaagacagcgacagtgtatgcacatatataaaataaataaatctttaaaaaacaaaacaagagagagggacatgctaccatttctacctcacttcttctcaaagccacccctaaagtgaattgtgaaccaggtcccctttgcagagagttagaagatattctcaaacctctaataccttcacatctaaaatccatcttcattccaaaattccaatattttatatacactctccagtttggtgggtgaggggttgttttttgtttggtttggtttggttggggttttgtttttgtttttgattttgtttttctctggttcagactccatggacgttcattaatgtcataaatgagttcattcc

SEQ ID NO: 4 - protein, 677 aa, homo sapiens
MAKCRVRVSTGEACGAGTWDKVSVSIVGTHGESPLVPLDHLGKEFSAGAEEDFEVTLPQDVGTVLMLRVHKAPPEVSLPLMSFRSDAWFCRWFELEWLPGAALHFPCYQWLEGAGELVLREGAAKVSWQDHHPTLQDQRQKELESRQKMYSWKTYIEGWPRCLDHETVKDLDLNIKYSAMKNAKLFFKAHSAYTELKVKGLLDRTGLWRSLREMRRLFNFRKTPAAEYVFAHWQEDAFFASQFLNGINPVLIRRCHSLPNNFPVTDEMVAPVLGPGTSLQAELEKGSLFLVDHGILSGVHTNILNGKPQFSAAPMTLLHQSSGSGPLLPIAIQLKQTPGPDNPIFLPSDDTWDWLLAKTWVRNSEFYIHEAVTHLLHAHLIPEVFALATLRQLPRCHPLFKLLIPHIRYTLHINTLARELLVAPGKLIDKSTGLGTGGFSDLIKRNMEQLNYSVLCLPEDIRARGVEDIPGYYYRDDGMQIWGAIKSFVSEIVSIYYPSDTSVQDDQELQAWVREIFSEGFLGRESSGMPSLLDTREALVQYITMVIFTCSAKHAAVSSGQFDSCVWMPNLPPTMQLPPPTSKGQARPESFIATLPAVNSSSYHIIALWLLSAEPGDQRPLGHYPDEHFTEDAPRRSVAAFQRKLIQISKGIRERNRGLALPYTYLDPPLIENSVSI

SEQ ID NO: 5 - DNA, 25 bp, homosapiens
gcgctgcggctctgggaaatcatct

SEQ ID NO: 6 - protein, 8 aa, homosapiens
ALRLWEII

SEQ ID NO: 7 - DNA, 25 bp, homosapiens
gggcccgaaaaatactcctcctcat

SEQ ID NO: 8 - protein, 8 aa, homosapiens
EEEYFSGP

SEQ ID NO: 9 - DNA, 22 bp, homosapiens
ctacccaagtgatgagtctgtc

SEQ ID NO: 10 - DNA, 23 bp, homosapiens
tgttcccctgggatttagatgga

(19) United States
(12) Patent Application Publication
(10) Pub. No.: US 2006/0217325 A1
(43) Pub. Date: Sep. 28, 2006

(54) Treatment of dry eye restoring 15-lipoxygenase activity to ocular surface cells

(75) Inventors: John M. Yanni, Burleson, TX (US); Daniel A. Gamache, Arlington, TX (US); Steven T. Miller, Arlington, TX (US)

Correspondence Address: Alcon Research, 6201 South Freeway, Fort Worth, TX, 76134-2099, US

(73) Assignee: Alcon, Inc., Hunenberg (CH)

(21) Appl. No.: 10/539,093

(22) PCT Filed: Oct. 17, 2003
(86) PCT No.: PCT/US03/33139

§ 371 Date: Jun. 15, 2005

Related U.S. Application Data
(60) Provisional application No. 60/435,988, filed on Dec. 20, 2002.

(51) Int. Cl. A61K 48/00

(52) U.S. Cl. 514/44; 424/93.2

(57) ABSTRACT

The present invention provides compositions comprising the 15-lipoxygenase-1 (15-LO-1) or 15-lipoxyngenase-2 (15-LO-2) gene such that 15-LO-1 or 15-LO-2 protein expression is replaced or replenished in the ocular surface epithelium of post-menopausal women suffering from dry eye. Thus, methods for treatment of dry eye in postmenopausal women are further provided.

This application claims priority from U.S. Ser. No. 60/435,988, filed Dec. 20, 2002.

### BACKGROUND OF THE INVENTION

1. Field of the Invention

The present invention relates to the field of dry eye. More particularly, the present invention relates to compositions and treatments for dry eye in post-menopausal women.

2. Description of the Related Art

Dry eye, also known generically as keratoconjunctivitis sicca, is a common ophthalmological disorder affecting millions of Americans each year. The condition is particularly widespread among post-menopausal women due to hormonal changes following the cessation of fertility. Dry eye may afflict an individual with varying severity. In mild cases, a patient may experience burning, a feeling of dryness, and persistent irritation such as is often caused by small bodies lodging between the eye lid and the eye surface. In severe cases, vision may be substantially impaired. Other diseases, such as Sjogren's disease and cicatricial pemphigoid manifest dry eye complications.

Although it appears that dry eye may result from a number of unrelated pathogenic causes, all presentations of the complication share a common effect, that is the breakdown of the pre-ocular tear film, which results in dehydration of the exposed outer surface and many of the symptoms outlined above (Lemp 1995).

Practitioners have taken several approaches to the treatment of dry eye. One common approach has been to supplement and stabilize the ocular tear film using so-called artificial tears instilled throughout the day. Other approaches include the use of ocular inserts that provide a tear substitute or stimulation of endogenous tear production.

Examples of the tear substitution approach include the use of buffered, isotonic saline solutions, aqueous solutions containing water soluble polymers that render the solutions more viscous and thus less easily shed by the eye. Tear reconstitution is also attempted by providing one or more components of the tear film such as phospholipids and oils. Phospholipid compositions have been shown to be useful in treating dry eye; see, e.g., McCulley and Shine (1998); and Shine and McCulley (1998). Examples of phospholipid compositions for the treatment of dry eye are disclosed in U.S. Pat. Nos. 4,131,651 (Shah et al.), 4,370,325 (Packian), 4,409,205 (Shively), 4,744,980 and 4,883,658 (Holly), 4,914,088 (Glonek), 5,075,104 (Gressel et al.), 5,278,151 (Korb et al.), 5,294,607 (Glonek et al.), 5,371,108 (Korb et al.), 5,578,586 (Glonek et al.). U.S. Pat. No. 5,174,988 (Mautone et al.) discloses phopholipid drug delivery systems involving phospholipids, propellants and an active substance.

United States Patents directed to the use of ocular inserts in the treatment of dry eye include U.S. Pat. No. 3,991,759 (Urquhart). Other semi-solid therapy has included the administration of carrageenans (U.S. Pat. No. 5,403,841, Lang) which gel upon contact with naturally occurring tear film.

Another approach involves the provision of lubricating substances in lieu of artificial tears. For example, U.S. Pat. No. 4,818,537 (Guo) discloses the use of a lubricating, liposome-based composition, and U.S. Pat. No. 5,800,807 (Hu et al.) discloses compositions containing glycerin and propylene glycol for treating dry eye.

Aside from the above efforts, which are directed primarily to the alleviation of symptoms associated with dry eye, methods and compositions directed to treatment of the dry eye condition have also been pursued. For example, U.S. Pat. No. 5,041,434 (Lubkin) discloses the use of sex steroids, such as conjugated estrogens, to treat dry eye condition in post-menopausal women; U.S. Pat. No. 5,290,572 (MacKeen) discloses the use of finely divided calcium ion compositions to stimulate preocular tear film; and U.S. Pat. No. 4,966,773 (Gressel et al.) discloses the use of microfine particles of one or more retinoids for ocular tissue normalization.

Although these approaches have met with some success, problems in the treatment of dry eye nevertheless remain. The use of tear substitutes, while temporarily effective, generally requires repeated application over the course of a patient's waking hours. It is not uncommon for a patient to have to apply artificial tear solution ten to twenty times over the course of the day. Such an undertaking is not only cumbersome and time consuming, but is also potentially very expensive.

The use of ocular inserts is also problematic. Aside from cost, they are often unwieldy and uncomfortable. Further, as foreign bodies introduced in the eye, they can be a source of contamination leading to infections. In situations where the insert does not itself produce and deliver a tear film, artificial tears must still be delivered on a regular and frequent basis.

Mucins are proteins which are heavily glycosylated with glucosamine-based moieties. Mucins provide protective and lubricating effects to epithelial cells, especially those of mucosal membranes. Mucins have been shown to be secreted by vesicles and discharged on the surface of the conjunctival epithelium of human eyes (Greiner et al., 1980; Dilly et al., 1981). A number of human-derived mucins which reside in the apical and subapical corneal epithelium have been discovered and cloned (Watanabe et al. 1995). Recently, Watanabe discovered a new mucin which is secreted via the cornea apical and subapical cells as well as the conjunctival epithelium of the human eye Watanabe et al. 1995). These mucins provide lubrication, and additionally attract and hold moisture and sebacious material for lubrication and the corneal refraction of light.

Mucins are also produced and secreted in other parts of the body including lung airway passages, and more specifically from goblet cells interspersed among tracheal/bronchial epithelial cells. Certain arachidonic acid metabolites have been shown to stimulate mucin production in these cells. Yanni reported the increased secretion of mucosal glycoproteins in rat lung by hydroxyeicosatetraenoic acid (“HETE”) derivatives (Yanni et al. 1989). Similarly, Marom has reported the production of mucosal glycoproteins in human lung by HETE derivatives (Marom et al. 1983).

Agents claimed for increasing ocular mucin and/or tear production include vasoactive intestinal polypeptide (Dartt et. al. 1996), gefarnate (Nakmura et. al. 1997), is liposomes (U.S. Pat. No. 4,818,537), androgens (U.S. Pat. No. 5,620,921), melanocycte stimulating hormones (U.S. Pat. No. 4,868,154), phosphodiesterase inhibitors (U.S. Pat. No. 4,753,945), and retinoids (U.S. Pat. No. 5,455,265). However, many of these compounds or treatments suffer from a lack of specificity, efficacy and potency and none of these agerits have been marketed so far as therapeutically useful products to treat dry eye and related ocular surface diseases.

U.S. Pat. No. 5,696,166 (Yanni et al.) discloses compositions containing naturally occurring HETEs, or derivatives thereof, and methods of use for treating dry eye. Yanni et al. discovered that compositions comprising HETEs increase ocular mucin secretion and are thus useful in treating dry eye.

In view of the foregoing, there is a need for an effective treatment for dry eye that is capable of alleviating symptoms, as well as treating the underlying physical and physiological deficiencies of dry eye, and that is convenient to administer.

### SUMMARY OF THE INVENTION

The present invention overcomes these and other drawbacks of the prior art by providing a method for treating dry eye by obtaining a composition comprising SEQ ID NO:1 or SEQ ID NO:3; and administering said composition to a patient suffering from dry eye under conditions such that SEQ ID NO:1 or SEQ ID NO:3 is expressed. In preferred embodiments, the composition for use in the invention comprises a vector comprising the sequence set forth in SEQ ID NO:1 or SEQ ID NO:3.

In another aspect, the invention provides a method of treating dry eye in a postmenopausal patient, by incorporating nucleic acid into an in situ ocular cell under conditions permissive for the uptake of the nucleic acid. Typically, the nucleic acid encodes a protein having the sequence set forth in SEQ ID NO:2 or in SEQ ID NO:4. Upon delivery to the ocular cell, the nucleic acid is expressed and the dry eye is thereby treated. Preferably, the nucleic acid sequence delivered to the patient will include the sequence set forth in SEQ ID NO:1. Typically, the cell is debrided prior to introducing the nucleic acid. It is anticipated that the nucleic acid may be incorporated into a viral vector, a plasmid, a retrovirus, an adenovirus, or an adeno-associated virus.

The present invention further provides a composition for treatment of dry eye. The composition of the invention includes a vector containing the sequence set forth in SEQ ID NO:1 or SEQ ID NO:3 and a pharmaceutically acceptable excipient.

### DETAILED DESCRIPTION PREFERRED EMBODIMENTS

Current therapies for dry eye focus on the use of wetting agents, such as artificial tears, to provide temporary relief of the condition. Additionally, punctal plugs may be surgically inserted to reduce the drainage of tears down the lacrimal duct. Neither method attempts to treat the cause of the symptoms associated with the disease, but rather treat the symptoms only. The present invention stems from the discovery that the ocular surface epithelium of postmenopausal women may lack 15-lipoxygenase (15-LO).

15-LO (SEQ ID NO:1) is a member of the lipoxygenase family, other members of which are found in a wide variety of mammalian and plant tissues. The corneal epithelia of many mammals contain significant activities of 15-LO and of 12-lipoxygenase (12-LO). Lipoxygenase metabolites of arachidonic acid (AA) and linoleic acid include e.g., hydroxyeicosatetraenoic acids (HETE), hydroperoxyeicosatetraenoic acids (HPETE), hydroxyoctadecadienoic acids, and hydroperoxyoctadecadienoic acids (Liminga and Oliw 2000).

Brash et al. (1997) discovered a second form of 15-LO (15-LO-2; SEQ ID NO:3) in humans. This second form of 15-LO at least partly accounts for the 15S-LO metabolism of arachidonic acid in certain epithelial tissues. Expression of 15-LO-2 was detected in human hair roots, prostate, lung and cornea. The cDNA (SEQ ID NO:3) encodes a protein of 676 amino acids (SEQ ID NO:4) with a calculated molecular mass of 76 kDa. Hsi et al. (2002) reported that an increased 15-LO-1 is positively associated with prostate cancer while 15-LO-2 is negatively associated with cancer.

The present inventors discovered that the ocular surface epithelium of postmenopausal women is lacking 15-LO. 15-LO is required for the synthesis of 15(S)-HETE, which in turn stimulates the production of MUC-1 mucin. According to the present invention, using an appropriate vector system, which will be readily available to the skilled artisan, the gene controlling the 15-LO-1 or 15-LO-2 enzyme is replaced or replenished in the ocluar surface epithelium of postmenopausal women suffering from dry eye.

Stechschulte et al. (2001) have shown that the cornea is readily accessible to gene therapy by injection of naked plasmid DNA into the cornea.

It has been shown that exogenous nucleic acid may be introduced into ocular cells, and in particular in situ ocular cells (U.S. Pat. No. 6,204,251). This may be accomplished by contacting an ocular cell with exogenous nucleic acid under conditions that allow the ocular cell to take up the exogenous nucleic acid into the ocular cell and express it. The present invention provides a method for introducing nucleic acid into an ocular cell such that the cell expresses the protein encoded by the nucleic acid. While the protein being expressed according to the present invention is an endogenous protein, it is under-expressed in ocular cells of postmenopausal women, thus resulting in dry eye. The compositions and methods of the present invention allow for increased expression of the under-expressed protein, thus providing treatment for the dry eye condition. This expression may be accomplished by means familiar to the skilled artisan, including, but not limited to, the methods described in U.S. Pat. No. 6,204,251, incorporated herein by reference.

The phrase “in situ ocular cell”, or grammatical equivalents, as used herein, refers to an ocular cell contained within the eye, i.e. in vivo. Ocular cells include cells of the lens, the cornea (both endothelial, stromal and epithelial corneal cells), the iris, the retina, choroid, sclera, ciliary body, vitrous body, ocular vasculature, canal of Schlemm, ocular muscle cells, optic nerve, and other ocular sensory, motor and autonomic nerves.

The term “nucleic acid”, or grammatical equivalents, as used herein, refers to either DNA or RNA, or molecules which contain both ribo- and deoxyribonucleotides.

It is understood that once a nucleic acid is made and reintroduced into a host cell or organism, it will replicate non-recombinantly, i.e. using the in vivo cellular machinery of the host cell rather than in vitro manipulations; however, such nucleic acids, once produced recombinantly, although subsequently replicated non-recombinantly, are still considered “recombinant” for the purposes of the present invention.

In the preferred embodiment, the nucleic acid introduced into the ocular cell, encodes a protein whose expression is desired to be increased. Typically, the nucleic acid will comprise the sequence of 15-LO-1 (SEQ ID NO:1), which encodes the 15-LO-1 protein (SEQ ID NO:2), or the sequence of 15-LO-2 (SEQ ID NO:3), which encodes the 15-LO-2 protein (SEQ ID NO:4). In certain preferred embodiments, the nucleic acid will encode from 27 to 2671 contiguous nucleotides of SEQ ID NO:1 or from 27 to 3224 contiguous nucleotides of SEQ ID NO:3. These shorter nucleic acids will encode peptide, polypeptide or protein sequences comprising from 9 to 661 contiguous amino acids from SEQ ID NO:2 or from 9 to 677 contiguous amino acids from SEQ ID NO:4. The skilled artisan can routinely determine active epitopes of the described nucleic acid sequences using techniques available in the art.

In an additional embodiment, the nucleic acid may encode a regulatory protein such as a transcription or translation regulatory protein. In this embodiment, the protein itself may not directly affect the ocular disease, but may instead cause the increase or decrease in the expression of another protein which affects the ocular disease.

The phrase “recombinant protein”, as used herein, refers to a protein made using recombinant techniques, i.e. through the expression of a recombinant nucleic acid as described above. A recombinant protein is distinguished from naturally occurring protein by at least one or more characteristics. For example, the protein may be made at a significantly higher concentration than is ordinarily seen, through the use of an inducible promoter or high expression promoter, such that increased levels of the protein are made.

The phrase “conditions permissive for the uptake of nucleic acid”, as used herein, refers to experimental conditions which allow the in situ ocular cell to take up, and be transformed with, the nucleic acid.

The permissive conditions will depend on the form of the nucleic acid. Thus, for example, when the nucleic acid is in the form of an adenoviral, retroviral, or adeno-associated viral vector, the permissive conditions are those which allow viral infection of the cell. Similarly, when the nucleic acid is in the form of a plasmid, the permissive conditions allow the plasmid to enter the cell. Thus, the form of the nucleic acid and the conditions which are permissive for its uptake are correlated. These conditions are generally well known in the art.

In a preferred embodiment, the nucleic acid encodes a protein that is expressed. In some embodiments, the expression of the nucleic acid is transient; that is, the protein is expressed for a limited time. In other embodiments, the expression is permanent.

In certain preferred embodiments, the nucleic acid is incorporated into the genome of the target cell; for example, retroviral vectors described below integrate into the genome of the host cell. Generally this is done when longer or permanent expression is desired. In other embodiments, the nucleic acid does not incorporate into the genome of the target cell but rather exists autonomously in the cell; for example, many such plasmids are known. This embodiment may be preferable when transient expression is desired.

Permissive conditions depend on the expression vector to be used, the amount of expression desired and the target cell. Generally, conditions which allow in vitro uptake of exogenous cells work for in vivo ocular cells. In some cases, the physical structural characteristics of the eye are taken into consideration.

For example, when the target cells are corneal epithelial cells, permissive conditions may include the debridement, or scraping of the corneal epithelium, in order to denude the corneal surface down to a basal layer of epithelium. The nucleic acid is then added, in a variety of ways as described below.

Permissive conditions are analyzed using well-known techniques in the art. For example, the expression of nucleic acid may be assayed by detecting the presence of mRNA, using Northern hybridization, or protein, using antibodies or biological function assays.

Specific conditions for the uptake of nucleic acid are well known in the art. They include, but are not limited to, retroviral infection, adenoviral infection, transformation with plasmids, transformation with liposomes containing nucleic acid, biolistic nucleic acid delivery (i.e. loading the nucleic acid onto gold or other metal particles and shooting or injecting into the cells), adeno-associated virus infection and Epstein-Barr virus infection. These may all be considered “expression vectors” for the purposes of the invention.

The expression vectors may be either extrachromosomal vectors or vectors which integrate into a host genome as outlined above. Generally, these expression vectors include transcriptional and translational regulatory nucleic acid operably linked to the nucleic acid. The phrase “operably linked,” as used herein, means that the transcriptional and translational regulatory DNA is positioned relative to the coding sequence of the protein to be expressed in such a manner that transcription is initiated. Generally, this will mean that the promoter and transcriptional initiation or start sequences are positioned 5′ to the coding region of the protein to be expressed. The transcriptional and translational regulatory nucleic acid will generally be appropriate to the ocular host cell used to express the protein; for example, transcriptional and translational regulatory nucleic acid sequences from mammalian cells, and particularly humans, are preferably used to express the desired protein in mammals and humans. Preferred are ocular cell transcriptional and translational regulatory sequences. Numerous types of appropriate expression vectors, and suitable regulatory sequences are known in the art.

In general, the transcriptional and translational regulatory sequences may include, but are not limited to, promoter sequence, ribosomal binding sites, transcriptional start and stop sequences, translational start and stop sequences, and enhancer or activator sequences. In a preferred embodiment, the regulatory sequences include a promoter and transcriptional start and stop sequences.

Promoter sequences encode either constitutive or inducible promoters. The promoters may be either naturally occurring promoters or hybrid promoters. Hybrid promoters, which combine elements of more than one promoter, are also known in the art, and are useful in the present invention.

In addition, the expression vector may comprise additional elements. For example, for integrating expression vectors, the expression vector contains at least one sequence homologous to the host cell genome, and preferably two homologous sequence which flank the expression construct. The integrating vector may be directed to a specific locus in the host cell by selecting the appropriate homologous sequence for inclusion in the vector. Constructs for integrating vectors are well known in the art.

The term “animal,” as used herein, refers to both humans and other animals and organisms. Thus, the methods of the present invention are applicable to both human therapy and veterinary applications.

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### EXAMPLE 1

The following experiments were performed by Dr. Alan Brash at Vanderbilt University Medical Center.

Tissues were processed, incubated and proteins extracted according to methods well known in the art. Reversed-phase HPLC was used to identify proteins extracted. RT-PCR was performed using methods well known in the art.

For RT-PCR analysis, two-thirds of the RNA was used to make first strand cDNA using oligo-dT as the primer. As a control, aliquots of each sample were incubated separately with and without reverse transcriptase.

15-LO-1 Primers:

“C4126” 15-lox-la upstream (amino acids ALRLWEII; SEQ ID NO:6)/“C4127” (amino acids EEEYFSGP; SEQ ID NO:8) 15-lox-la downstream.”

Upstream nucleic acid sequence:

(SEQ ID NO:5)

5′-GCG-CTG-CGG-CTC-TGG-GAA-ATC-ATC-T
Downstream nucleic acid sequence:

(SEQ ID NO:7)

5′-GG-GCC-CGA-AAA-ATA-CTC-CTC-CTC-AT

15-LO-2 Primers:

“DESV upstream/SI* downstream”

Upstream nucleic acid sequence:

(SEQ ID NO:9)

5′-C-TAC-CCA-AGT-GAT-GAG-TCT-GTC
Downstream nucleic acid sequence:

(SEQ ID NO:10)

5′-TGTTCCCCTGGGAT-TTA-GAT-GGA

Western analyses was performed using rabbit polyclonal anti-human 15-LO-2 antibody, prepared against purified 15-LO-2 protein as described (Shappell et al. 1999). Previous western blot studies have shown that this antibody binds strongly to 15-LO-2, without cross reactivity to 15-LO-1,5-LO or platelet type 12-LO, and with weak cross-reactivity to human 12R-LO (Shappell et al. 1999). In Western analyses, this antibody will detect 15-LO-2 protein in prostate and cornea. Antibody to 15-LO-1 was a gift from Dr. Joseph Cornicelli (Parke-Davis); this polyclonal antibody to human 15-LO-1 was raised in goats. The Alkaline Phosphatase/Nitro Blue Tetrazolium method was used for detection.

For reversed-phase HPLC (RP-HPLC) analyses of conjunctiva samples, the homogenized tissue was incubated with ¹⁴C-arachidonic acid substrate and extracted. The radioactivity associated with any products was measured by RP-HPLC with an on-line ¹⁴C detector. An aliquot of the UV-absorbing standards were mixed with every sample and detected using a diode array UV detector at the same time. This compensated for any slight differences in retention times of authentic HETEs from run to run or day to day. The results showed activity of 15-LO in 7 of 21 samples, with weak to negative activity in 12 samples.

From four of the samples that were positive for 15-LO activity, RNA samples were prepared and aliquots tested ± reverse transcriptase using separate primer sets known to polymerize 15-LO-1 or 15-LO-2. The RNA samples not treated with reverse transcriptase acted as control against possible contamination with authentic 15-LO cDNAs. The results of this qualitative test on four samples showed one positive for 15-LO-1, and one sample positive for both LO isozymes. This method serves as a guide for the possible presence of the expressed enzymes in conjunctiva.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied is to the compositions and/or methods and in the steps or in the sequence of steps of the method described herein without departing from the concept, spirit and scope of the invention. More specifically, it will be apparent that certain agents which are both chemically and structurally related may be substituted for the agents described herein to achieve similar results. All such substitutions and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.

### UNITED STATES PATENT

U.S. Pat. No. 3,991,759
U.S. Pat. No. 4,131,651
U.S. Pat. No. 4,370,325
U.S. Pat. No. 4,409,205
U.S. Pat. No. 4,744,980
U.S. Pat. No. 4,753,945
U.S. Pat. No. 4,818,537
U.S. Pat. No. 4,868,154
U.S. Pat. No. 4,883,658
U.S. Pat. No. 4,914,088
U.S. Pat. No. 4,966,773
U.S. Pat. No. 5,041,434
U.S. Pat. No. 5,075,104
U.S. Pat. No. 5,174,988
U.S. Pat. No. 5,278,151
U.S. Pat. No. 5,290,572
U.S. Pat. No. 5,294,607
U.S. Pat. No. 5,371,108
U.S. Pat. No. 5,403,841
U.S. Pat. No. 5,455,265
U.S. Pat. No. 5,578,586
U.S. Pat. No. 5,620,921
U.S. Pat. No. 5,696,166
U.S. Pat. No. 5,800,807
U.S. Pat. No. 6,204,251

### OTHER PUBLICATIONS

Brash et al., Proc. Natl. Acad. Sci. USA 94:6148-6152 (1997).
Dartt et. al., Experimental Eye Research 63:27-34, (1996).
Dilly et al., British Journal of Ophthalmology 65:833-842 (1981).
Greiner et al., Archives of Ophthalmology 98:1843-1846 (1980).
Hsi et al., Prostaglandins Leukot. Essent. Fatty Acids 64:217-225 (2001).
Hsi et al., JBC 277(43):40549-40556 (2002).
Lemp, Report of the Nation Eve Institute/Industry Workshop on Clinical Trials in Dry Eyes, The CLAO Journal, 21(4):221-231 (1995).
Liminga and Oliw, Lipids 35(2):225-232 (2000).
Marom et al., Journal of Clinical Investigation 72:122-127 (1983).
McCulley and Shine, Contactologia, 20(4):145-49 (1998).
Nakmura et. al., Experimental Eye Research 65:569-574 (1997).
Shappell et al., Amer. J. Path. 155:235-245 (1999).
Shappell et al., J. Invest. Dermatol. 117:36-43 (2001).
Shine and McCulley, Archives of Ophthalmology 116(7):849-52 (1998).
Stechshulte et al., Invest. Ophthalmol. Vis. Sci. 42(9):1975-1979 (2001).
Watanabe et al., Investigative Ophthalmology and Visual Science 36(2):337-344 (1995).
Yanni et al, International Archives of Allergy And Applied Immunology 90:307-309 (1989).

We claim:

1. A method for treating dry eye, said method comprising:
a) obtaining a composition comprising SEQ ID NO:1 or SEQ ID NO:3; and
b) administering said composition to a patient suffering from dry eye under conditions such that SEQ ID NO:1 or SEQ ID NO:3 is expressed.

2. The method of claim 1, wherein said composition comprises a vector comprising the sequence set forth in SEQ ID NO:1 or SEQ ID NO:3

3. The method of claim 1, wherein said administering is by topical ocular drops or ointment.

4. A composition for treatment of dry eye, said composition comprising a vector comprising the sequence set forth in SEQ ID NO:1 or SEQ ID NO:3 and a pharmaceutically acceptable excipient.

5. A method of treating dry eye in a postmenopausal patient, said method comprising incorporating nucleic acid into an in situ ocular cell under conditions permissive for the uptake of said nucleic acid, said nucleic acid encoding a protein having the sequence set forth in SEQ ID NO:2, whereby said nucleic acid is expressed and said disease is treated.

6. The method of claim 6, wherein said nucleic acid sequence comprises the sequence set forth in SEQ ID NO:1.

7. The method of claim 7, wherein said cell is a conjunctival or corneal epithelial cell.

8. The method of claim 8, wherein said cell is debrided prior to introducing said exogenous nucleic acid.

9. The method of claim 6, wherein said nucleic acid is in a viral vector.

10. The method of claim 6, wherein said nucleic acid is in a plasmid.

11. The method of claim 10, wherein said nucleic acid is in a retrovirus.

12. The method of claim 10, wherein said nucleic acid is in an adenovirus.

13. The method of claim 10, wherein said nucleic acid is in an adeno-associated virus.

14. A method of treating dry eye in a postmenopausal patient, said method comprising incorporating nucleic acid into an in situ ocular cell under conditions permissive for the uptake of said nucleic acid, said nucleic acid encoding a protein having the sequence set forth in SEQ ID NO:4, whereby said nucleic acid is expressed and said disease is treated.

15. The method of claim 15, wherein said nucleic acid sequence comprises the sequence set forth in SEQ ID NO:3.

16. The method of claim 16, wherein said cell is a conjunctival or corneal epithelial cell.

17. The method of claim 7, wherein said cell is debrided prior to introducing said exogenous nucleic acid.

18. The method of claim 15, wherein said nucleic acid is in a viral vector.

19. The method of claim 15, wherein said nucleic acid is in a plasmid.

20. The method of claim 19, wherein said nucleic acid is in a retrovirus.

21. The method of claim 19, wherein said nucleic acid is in an adenovirus.

22. The method of claim 19, wherein said nucleic acid is in an adeno-associated virus.
